(19) United States
(12) Patent Application Publication
(10) Pub. No.: US 2020/0342953 A1
(43) Pub. Date: Oct. 29, 2020

(54) TARGET MOLECULE-LIGAND BINDING MODE PREDICTION COMBINING DEEP LEARNING-BASED INFORMATICS WITH MOLECULAR DOCKING

(71) Applicant: INTERNATIONAL BUSINESS MACHINES CORPORATION, ARMONK, NY (US)

(72) Inventors: Joseph Anthony Morrone, White Plains, NY (US); Jeffrey Kurt Weber, Brooklyn, NY (US); Wendy Dawn Cornell, Warren, NJ (US)

(21) Appl. No.: 16/397,003

(22) Filed: Apr. 29, 2019

(51) Int. Cl. G16B 15/30; G16B 45/00; G16B 20/30; G16B 40/00; G16B 5/00; G06N 3/08

(52) U.S. Cl. CPC G16B 15/30; G16B 45/00; G06N 3/08; G16B 40/00; G16B 5/00; G16B 20/30

(57) ABSTRACT

A computer-implemented method is described. The method includes generating, by a ligand bond graph generator, a first graph based on bond connectivity within a ligand molecule that is specified as input. The method further includes generating, by a ligand-protein graph generator, a second graph based on a contact map of the ligand molecule and a target molecule that is specified as another input. The method further includes receiving docking prediction metrics for the ligand molecule and the target molecule. The method further includes inputting, to a deep neural network, as input features, the first graph, the second graph, and the docking prediction metrics. The method further includes determining, using the deep neural network, a binding mode prediction that characterizes a set of potential interactions between the ligand molecule and the target molecule.

### BACKGROUND

The present invention relates in general to computational chemistry and modeling, drug discovery, and particularly to combining machine learning techniques and molecular docking techniques for predicting target molecule-ligand binding.

Traditionally, drug discovery has been an expensive, multi-step, multi-year long process. Computational drug discovery has, therefore, gained importance. Small molecule lead finding efforts now leverage virtual screening techniques, in which potential drug molecules/ligands (leads) are ranked according to their predicted affinities for a protein (or other) target(s). In silico protocols aid in both the optimization of lead compounds and the understanding of drug mechanisms of action.

### SUMMARY

A computer-implemented method is described. The method includes generating, by a ligand bond graph generator, a first graph based on bond connectivity within a ligand molecule that is specified as input. The method further includes generating, by a ligand-protein graph generator, a second graph based on a contact map of the ligand molecule and a target molecule that is specified as another input. The method further includes receiving docking prediction metrics for the ligand molecule and the target molecule. The method further includes inputting, to a deep neural network, as input features, the first graph, the second graph, and the docking prediction metrics. The method further includes determining, using the deep neural network, a binding mode prediction that characterizes a set of potential interactions between the ligand molecule and the target molecule.

According to one or more embodiments, a system includes a memory, and a processor coupled with the memory. The processor performs a method for providing binding mode predictions for one or more ligand molecules and a target molecule. The method includes generating a first graph based on bond connectivity within a ligand molecule that is specified as input. The method further includes generating a second graph based on a contact map of the ligand molecule and a target molecule that is specified as another input. The method further includes receiving docking prediction metrics for the ligand molecule and the target molecule. The method further includes inputting, to a deep neural network, as input features, the first graph, the second graph, and the docking prediction metrics. The method further includes determining, using the deep neural network, a binding mode prediction that indicates a set of potential interactions between the ligand molecule and the target molecule.

According to one or more embodiments of the present invention, a computer program product includes a memory storage device having computer executable instructions stored therein, the computer executable instructions when executed by a processor cause the processor to perform a method for providing binding mode predictions for one or more ligand molecules and a target molecule. The method includes generating a first graph based on bond connectivity within a ligand molecule that is specified as input. The method further includes generating a second graph based on a contact map of the ligand molecule and a target molecule that is specified as another input. The method further includes receiving docking prediction metrics for the ligand molecule and the target molecule. The method further includes inputting, to a deep neural network, as input features, the first graph, the second graph, and the docking prediction metrics. The method further includes determining, using the deep neural network, a binding mode prediction that indicates a set of potential interactions between the ligand molecule and the target molecule.

Additional technical features and benefits are realized through the techniques of the present invention. Embodiments and aspects of the invention are described in detail herein and are considered a part of the claimed subject matter. For a better understanding, refer to the detailed description and to the drawings.

### BRIEF DESCRIPTION OF THE DRAWINGS

The examples described throughout the present document will be better understood with reference to the following drawings and description. The components in the figures are not necessarily to scale. Moreover, in the figures, like-referenced numerals designate corresponding parts throughout the different views.

FIG. 1 depicts a block diagram of the molecular interaction system according to one or more embodiments of the present invention;

FIG. 2 depicts examples of graphs generated according to one or more embodiments of the present invention;

FIG. 3 depicts an example multi-layer convolutional neural network (CNN) according to one or more embodiments of the present invention;

FIG. 4 depicts an example deep neural network (DNN) used by one or more embodiments of the present invention;

FIG. 5 depicts an example node from a DNN according to one or more embodiments of the present invention; and

FIG. 6 depicts a flowchart of a method for target molecule-ligand binding mode prediction by combining deep learning-based informatics with molecular docking according to one or more embodiments of the present invention.

The diagrams depicted herein are illustrative. There can be many variations to the diagram or the operations described therein without departing from the spirit of the invention. For instance, the actions can be performed in a differing order or actions can be added, deleted or modified. Also, the term “coupled” and variations thereof describes having a communications path between two elements and does not imply a direct connection between the elements with no intervening elements/connections between them. All of these variations are considered a part of the specification.

### DETAILED DESCRIPTION

Various embodiments of the invention are described herein with reference to the related drawings. Alternative embodiments of the invention can be devised without departing from the scope of this invention. Various connections and positional relationships (e.g., over, below, adjacent, etc.) are set forth between elements in the following description and in the drawings. These connections and/or positional relationships, unless specified otherwise, can be direct or indirect, and the present invention is not intended to be limiting in this respect. Accordingly, a coupling of entities can refer to either a direct or an indirect coupling, and a positional relationship between entities can be a direct or indirect positional relationship. Moreover, the various tasks and process steps described herein can be incorporated into a more comprehensive procedure or process having additional steps or functionality not described in detail herein.

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any embodiment or design described herein as “exemplary” is not necessarily to be construed as preferred or advantageous over other embodiments or designs. The terms “at least one” and “one or more” can be understood to include any integer number greater than or equal to one, i.e. one, two, three, four, etc. The terms “a plurality” can be understood to include any integer number greater than or equal to two, i.e. two, three, four, five, etc. The term “connection” can include both an indirect “connection” and a direct “connection.”

The terms “about,” “substantially,” “approximately,” and variations thereof, are intended to include the degree of error associated with measurement of the particular quantity based upon the equipment available at the time of filing the application. For example, “about” can include a range of ±8% or 5%, or 2% of a given value.

For the sake of brevity, conventional techniques related to making and using aspects of the invention may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

Developing accurate and reliable methods for binding mode prediction is a critical technical problem in computational chemistry, particularly when used for small molecule lead finding wherein potential drug molecules/ligands (leads) are ranked according to their predicted affinities for a protein (or other) target(s), such as during drug discovery. In computational structure-based drug design, binding mode prediction is a foundational part of virtual screening workflows. Lead optimization is difficult to perform, and atomistic mechanisms of action are hard to elucidate without first generating accurate binding modes. Therefore, accurate prediction of binding modes between a target molecule (e.g., a biopolymer such as a protein or nucleic acid) and its ligand(s) (any molecule that potentially binds to the target) is a key part of computational and structure-based drug design. Embodiments of the present invention address such technical problems and improve the prediction of target-ligand binding modes by combining predictions from small molecule docking tools and target-ligand cheminformatics within a unified deep learning framework.

One or more embodiments of the present invention improve the prediction by combining informatic, machine learning (or deep learning) generated features of a ligand and corresponding molecular target-ligand complex with features output by docking programs, using another machine learning system for binding mode prediction. Technical problems in such combining of information are addressed by one or more embodiments of the present invention by applying a graph convolutional technique to a contact map of target-ligand interactions to facilitate binding mode prediction. Ligand and target sites are determined to be in contact with each other based on a distance cutoff. Graph edges can be weighted according to the value of the distance. One or more embodiments of the present invention use a convolutional neural network to generate an internal representation of the graph which is then used for the prediction.

According to one or more embodiments of the present invention, molecular interaction prediction is implemented in one or more forms, such as: a system, a method, a computer program product, and the like. By addressing one or more technical problems with existing technical solutions for molecular interaction prediction, one or more embodiments of the present invention improve the performance of molecular interaction prediction. As will be evident based on the description herein, the technical solutions described herein are practical applications at least in the field of computational chemistry, drug discovery, molecular interaction, and the like.

Presently available computational drug discovery efforts rely on both physics-based and data-based machine learning/informatics approaches. In existing systems, a typical paradigm for making predictions (e.g. identifying leads) is based on either 3-dimensional (3D) protein-ligand complex coordinates or simple ligand chemical structures. In the latter case, traditional cheminformatics is often used to generate a “fingerprint” representation before machine learning is applied. Such techniques then use databases in combination with machine learning to predict ligand activity and other ligand properties. In the case of the 3D protein-ligand complex based systems, physics-based docking programs are used. “Docking programs” are the standard tool for binding mode prediction and typically employ physics based or statistical methodologies to simulate target-ligand interactions in molecular detail.

One or more embodiments of the present invention improve such existing techniques by combining different approaches, under a deep learning framework, to yield unified predictions of experimental binding modes. Further, for facilitating such combinations, embodiments of the present invention use bond connectivity within the ligand molecule to generate a first graph. Further, a second graph (referred herein as a “contact-map”) is generated from the 3D protein-ligand structure by computing the distances between ligand and protein sites. Edges in the second graph can be weighted according to the value of the distances between ligand and protein sites or assigned unit values based on a distance cutoff. In addition, docking prediction metric(s) from a docking program are also used. The first graph, the second graph, and the docking metrics are combined and used as feature(s) that are input into a first neural network. It should be noted that the first and second graphs are generated using respective neural network in one or more embodiments of the present invention. The first neural network uses the graphs and dense/input layers for creating physically derived concatenated features from these inputs. The combined representation is further fed into a subsequent deep neural network (DNN). From the output layer of the DNN, a prediction of whether the candidate binding modes are experimentally realizable is predicted. This prediction can be made, for example, using a binary classifier with a soft-max output final layer. A detailed description of the various one or more components, systems, apparatus, and methods used to achieve these results are now described in further detail.

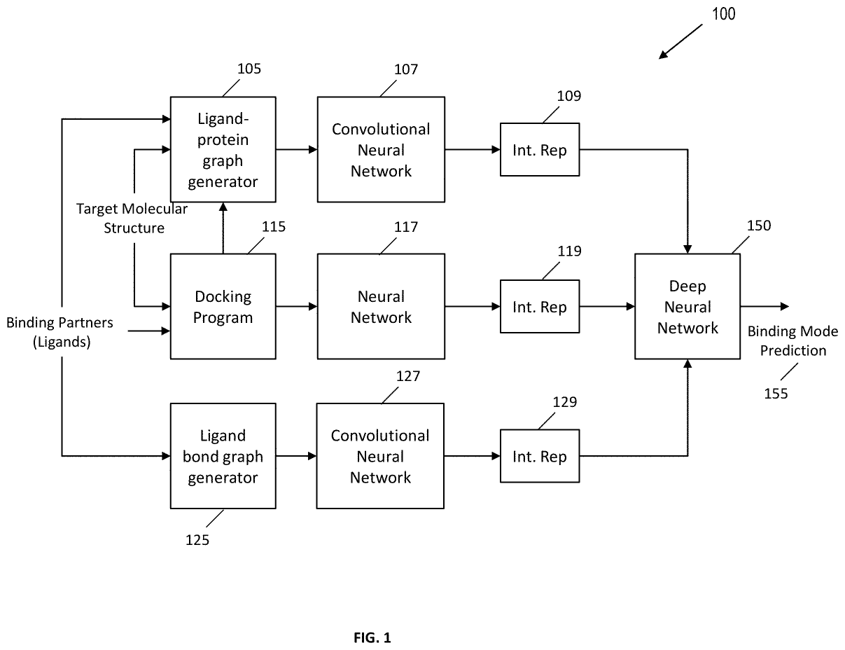

FIG. 1 depicts a block diagram of the molecular interaction system 100 according to one or more embodiments of the present invention. The system 100 includes a ligand-protein graph generator 105, a docking program 115, and a ligand bond graph generator 125. The system 100 further includes one or more convolutional neural networks (CNNs). The CNNs include a first CNN 107 associated with the ligand-protein graph generator 105. The CNNs further include a second CNN 117 that is associated with the ligand bond graph generator 125. Further, a neural network 117 is associated with the docking program 115.

In the system 100 protein or other molecular target structure(s) and possible binding partners (ligands) are provided as input. The system 100 provides, as final output 155, a binding mode prediction. Here, “binding modes” are defined as three-dimensional representations of the atomic coordinates of the target molecule interacting with the ligand molecule. Binding modes can be characterized using a root-mean square deviation (RMSD) metric which measures the difference (in units of length) of the ligand's orientation and position relative to the target with respect to a reference structure. Accordingly, a “binding mode prediction” in the context herein is a prediction of the characterization of the sampled complexes (3D structures) formed between a ligand molecule and a target molecule (i.e., binding modes or poses). It should be noted that the binding mode prediction herein is not limited to the generation/sampling of binding modes but is also a prediction of their ‘character’ as related to ligand RMSD or other such metrics.

The docking program 115 generates intermediate binding mode predictions. Typically, the docking programs 115 sample and rank binding modes according to a docking scoring function (sometimes referred to as an energy). It should be noted that docking programs are known tools in computational drug discovery, and are known to be limited in their accuracy. The docking scores generated by the docking programs 115 assess the experimental feasibility of each binding mode sampled by the docking programs 115. In one or more examples, the intermediate binding mode prediction metrics can include the pose ranking or the raw pose score (energy).

Such intermediate binding mode predictions are used as inputs to the neural network 117. The neural network 117 generates an internal representation 119 of the intermediate binding mode predictions based on the initial weights configured in the neural network 117. The internal representation 119 is the output layer of the neural network 117, in one or more examples.

Further, the ligand bond graph generator 125 uses bond connectivity within the ligand molecule to generate a first graph. Additionally, the Ligand-protein graph generator 105 uses the 3-dimensional structure of the ligand molecule and of the target molecule, and the docking scores from the docking program 115 to generate a second graph.

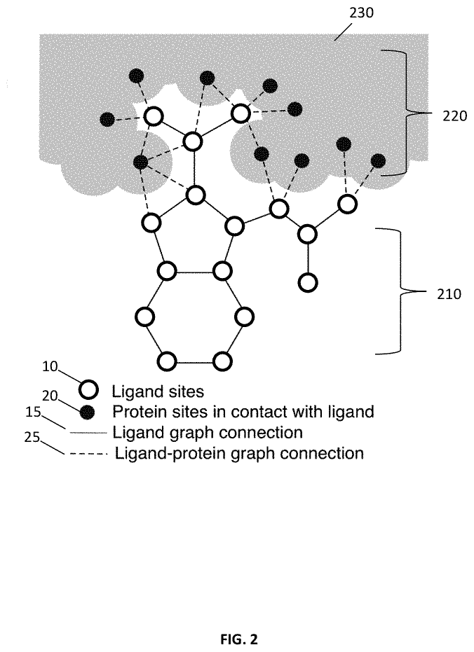

FIG. 2 depicts examples of graphs generated according to one or more embodiments of the present invention. As shown in FIG. 2, the system 100, based on the 3D structure of the target-ligand complex, generates two graphs. In first graph 210, ligand sites 10 (white circles) are connected to each other according to chemical bonds 15 (solid lines). The ligand bond graph generator 125 uses bond connectivity within the ligand molecule to generate the first graph 210. In the second graph 220, protein sites 20 (black circles) in contact with the ligand sites 10 form a separate graph connected by ligand-protein edges 25 (dashed lines). The protein surface 230 is shown that includes the protein sites 20.

The ligand-protein graph generator 105 generates the second graph 220 based on the ligand sites 10 and protein sites 20. A pair of a ligand site 10 and a protein site 20 is determined to be in contact with each other based on a distance cutoff. For example, if the distance between the ligand site 10 and the protein site 20 is below a predetermined threshold, the pair of sites is deemed to be in contact. An edge 25 of the second graph 220 can also be weighted according to the value of the distance between the ligand site 10 and the protein site 20 connected by the edge 25.

Subsequently, the system 100 utilizes the CNNs 107 and 127 to generate an internal representation 109 of the first graph 210 and an internal representation 129 of the second graph 220 respectively. Using CNNs for generating the internal representations 109 and 129 allows the system 100 to learn position and scale invariant structures in the input molecular structure data.

The first graph 210 and the second graph 220 each have vertices representing the sites (10, 20) and edges (15, 25) representing relationships between the vertices. The graphs (210 and 220) contain at least two forms of information: relational knowledge and categorical knowledge. Relational knowledge encodes the relationship between the sites (10, 20), while categorical knowledge encodes the attributes of the sites (10, 20). The graphs (210, 220) can be stored electronically, such as using files, databases, or other data structures in volatile/non-volatile memory. The graphs (210, 220) can be input to the respective CNNs (107, 127) for generating the corresponding internal representations. The CNNs (107, 127) incorporate hard constraints on learning and are good at detecting invariants on data, either to translation or deformation. They use three basic concepts: i) local receptive fields, ii) weight sharing, and iii) spatial subsampling.

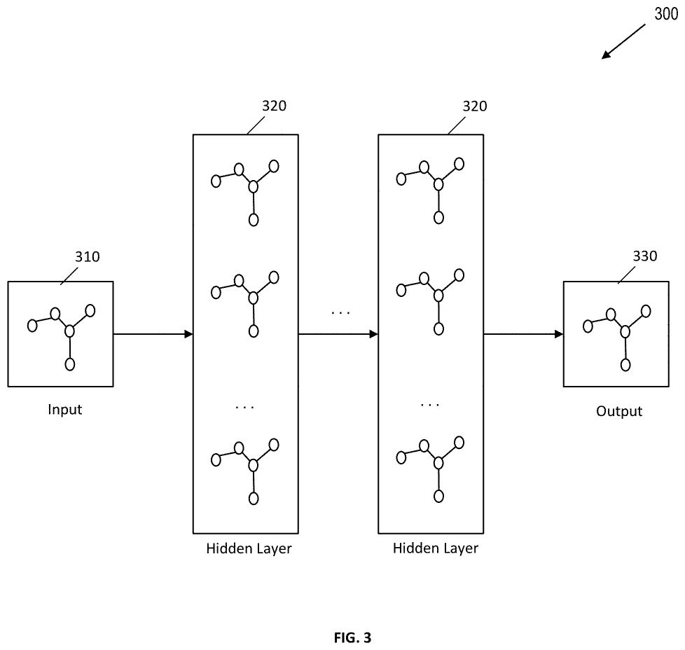

FIG. 3 depicts an example multi-layer CNN according to one or more embodiments of the present invention. The depicted CNN 300 is representative of both, the CNN 107 and the CNN 127. Each of the CNNs (107, 127) can be a graph convolutional network (GCN) in one or more examples. In one or more embodiments of the present invention, the CNNs (107, 127) make use of graph convolutions known from spectral graph theory to define parameterized filters that are used in a multi-layer CNN model. Accordingly, for the CNNs (107, 127), the goal is then to learn a function of signals/features on the input graph 310 G=(V, E), which can be the first graph 210 or the second graph 220. The function takes as input: (i) a feature description xi for every node i; summarized in a N×D feature matrix X (N: number of nodes, D: number of input features); and (ii) a representative description of the graph structure in matrix form; typically in the form of an adjacency matrix A (or some function thereof). The function produces a node-level output 330 Z (an N×F feature matrix, where F is the number of output features per node). In one or more examples, graph-level outputs can be modeled by introducing some form of pooling operation.

Accordingly, each neural network layer 320 can then be written as a non-linear function H(l+1)=f(H(l),A), with H(0)=X and H(l)=Z (or z for graph-level outputs), L being the number of layers. The specific models then differ only in how f(⋅,⋅) is chosen and parameterized. Each layer 320 (referred to as “hidden” layer) is further associated with a weight matrix, for example, W(l) for the l-th neural network layer 320. In one or more examples, the non-linear activation function can be the Rectified Linear Units (ReLU) function. It should be noted that although FIG. 3 depicts two hidden layers 320, in one or more examples, the number of hidden layers 320 can be any other integer. The CNNs (107, 127) can be pre-trained using supervised learning, semi-supervised learning, or any other techniques. Accordingly, the CNNs (107, 127) generate the internal representations (109, 129) of the first graph 210 and the second graph 220, respectively.

Referring to FIG. 1, the docking scores or metrics generated by the docking program 115 are fed into the neural network 117 to generate an internal representation 119.

Here, the “internal representation” is a vector in state space that represents a set of states (input, inner, or output states) of a unit for many sample data, in this case the input ligand sites 10 and protein sites 20. Internal representations (109, 119, and 129) are symbolic representations of the respective input data that use a predetermined common symbol language, in which an agent can express and manipulate propositions about the world. For symbolic representations that are languages of logic, however, some preparations have to be made central for reasoning about internal representation and symbol manipulation. In one or more examples, the internal representation can also be known as first order logic (FOL). In other words, “internal representation” is the output vector of a hidden layer at a given point in the CNNs (107, 117, and 127). The vector is represented in a basis determined by the prior layers which when trained should learn pertinent features.

Referring to FIG. 1, all internal representations (109, 119, and 129) are combined (concatenated) after applying the neural networks (107, 117, and 127). This combined representation is fed into the DNN 150. Any known technique for implementing a DNN can be used for the DNN 150.

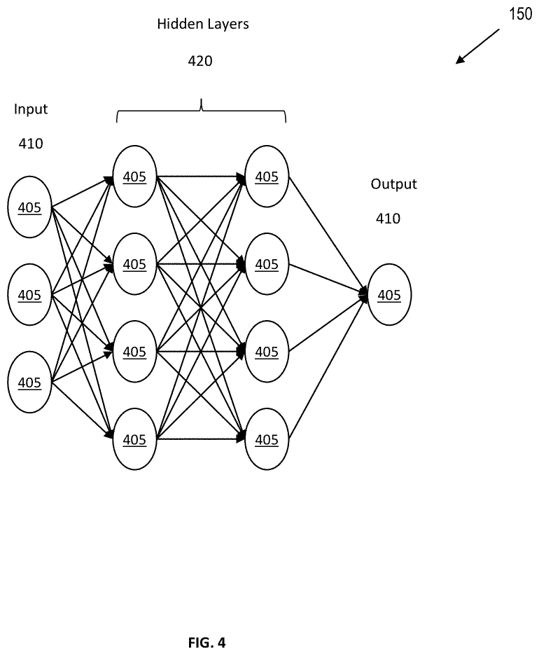

FIG. 4 depicts an example DNN used by one or more embodiments of the present invention. The DNN 150 includes multiple (two or more) layers of neurons 405, where each layer of nodes trains on a distinct set of features based on the previous layer's output. The further (to the right in FIG. 4) the layer in the DNN 150, the more complex the features the nodes can recognize, because the layers aggregate and recombine features from the previous layer(s). This feature hierarchy of increasing complexity and abstraction makes the DNN 150 capable of handling very large, high-dimensional data sets with billions of parameters that pass through nonlinear functions. The DNN 150 in this manner is capable of discovering latent structures within unlabeled, unstructured data that is input in the input layer 410.

When training on unlabeled data, each node layer 420 in the DNN 150 learns features automatically by repeatedly trying to reconstruct the input from which it draws its samples, attempting to minimize the difference between the network's guesses and the probability distribution of the input data itself. Restricted Boltzmann machines, for examples, create so-called reconstructions in this manner. In the process, the DNN 150 can learn to recognize correlations between certain relevant features and optimal results—by drawing connections between feature signals and what those features represent, whether it be a full reconstruction, or with labeled data. The DNN 150 ends in an output layer 430: a logistic, or softmax, classifier that assigns a likelihood to a particular outcome or label. The likelihood is referred to as the “predictive score” or the binding mode prediction 155. Given raw data in the form of the ligands and target molecular structure, the predictive system using the DNN 150 determines that the input ligands are P % likely to bind with the target molecular structure.

It should be noted that although the DNN 150 is shown to include only two hidden layers 420, in other examples, the DNN 150 can include any number of hidden layers 420 greater than two.

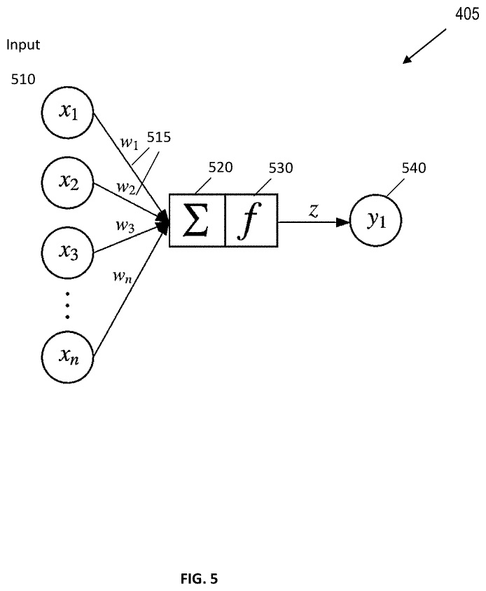

FIG. 5 depicts an example node from a DNN according to one or more embodiments of the present invention. Any node 405 takes the weighted sum 520 of its inputs 510, and passes the weighted sum through a non-linear activation function 530 (e.g. f). The weighted sum 520 uses weights 515 associated with each input edge. The weights are initiated and dynamically updated during training. The output of the non-linear activation function 530 is the output 540 of the node 405, which then becomes the input of another node in the next layer. The signal flows from left to right in FIG. 4, and the final output 430 is calculated by performing this procedure for all the nodes 405. Training the DNN 150 means learning the weights associated with all the edges. The DNN 150 can be trained using any known technique such as backpropagation with gradient descent.

In one or more embodiments of the present invention, from the output layer 530, a prediction of whether the candidate binding modes are experimentally realizable is extracted. This prediction can be made using a binary classifier with a soft-max output layer 530. The predictions can be compared directly with experimental data (e.g. X-ray crystal or NMR structures).

Accordingly, referring to FIG. 1, the system 100 determines the binding mode prediction 155 that the input ligands are P % likely to bind with the target molecular structure that are input to the system using the combination of the data from the various components described herein.

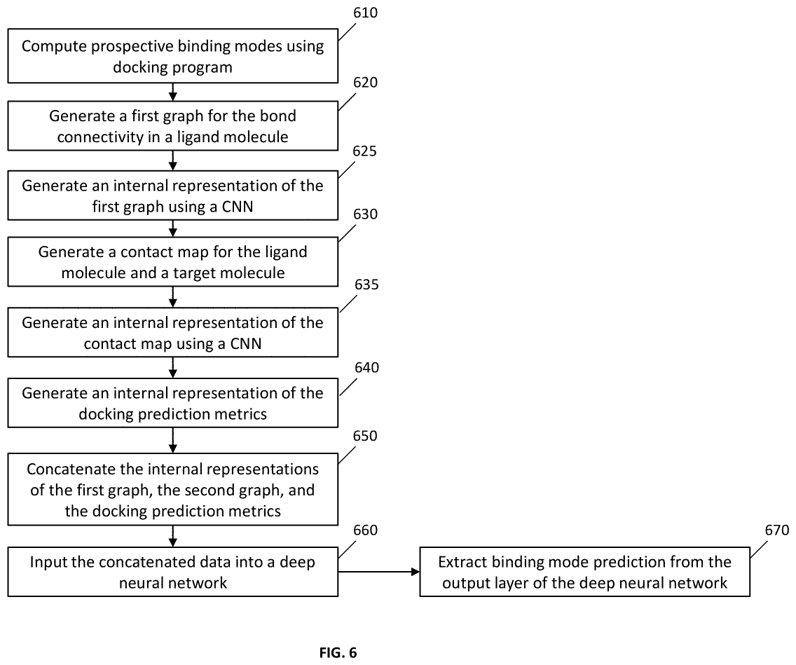

FIG. 6 depicts a flowchart of a method for target molecule-ligand binding mode prediction by combining deep learning-based informatics with molecular docking according to one or more embodiments of the present invention. The method includes generating prospective binding modes using methods such as docking programs 115 for specified input data, at 610. The input data includes a protein or other molecular target structure(s) and possible binding partners (ligands). The input data can be provided electronically, such as in the form of digital files, database, or any other such digital format.

Further, the method includes using bond connectivity within the ligand molecule to generate the first graph 210, at 620. The ligand graph generator 125 can generate the first graph 210. The method further includes, using the CNN 107 to generate the internal representation 109 of the first graph 210, at 625.

The method further includes generating the internal representation 129 based on the second graph 220, which is turn is based on target-ligand contacts in three-dimensions, at 630 and 635. The ligand-protein graph generator 105 generates the second graph 220. This includes generating the 3D contact map of target-ligand interactions to facilitate binding mode prediction. Generating the contact map includes determining whether the ligand and target sites (10, 20) are in contact with each other using a distance cutoff. The pairs of ligand and target sites (10, 20) in the contact map are connected using graph edges that can be weighted according to the value of the distance in the pair. The CNN 127 is then used to generate the internal representation 129 of this contact map.

Further, the method includes using the docking prediction metric(s) as feature(s) that are fed into the neural network 117 to generate the internal representation 119, at 640.

The internal representations (109, 119, and 129) are combined (concatenated) after applying the CNNs to the graphs (107, 109) and dense/input layers to the docking derived features from the docking program 105, at 650. The combined representation is input into the DNN 150, at 660. From the output layer, a prediction of whether the candidate binding modes are experimentally realizable along with confidence values of said predictions are extracted, at 670. The prediction is provided as the binding mode prediction 155.

Binding mode prediction 155 that is output can be utilized/applied for lead finding or lead optimization by identifying molecules predicted to bind to on-targets or off-targets in various ways. For example, the binding mode prediction 155 can be used for docking where the molecule is scored and ranked using the predicted pose as input. Further, the binding mode prediction 155 can be used for performing a 3D similarity search using the predicted pose as input. Further yet, the binding mode prediction 155 can be used for 3D pharmacophore generation for a target family using predicted poses as input. In addition, or alternatively, the binding mode prediction 155 can be used for 3D pharmacophore generation for an individual target using predicted poses as input. The binding mode prediction 155 can also be used for small molecule library design via R group substitution using the predicted pose as input. Alternatively yet, generative drug design using the predicted pose as input can be performed based on the binding mode prediction 155. Refinement of an experimental protein structure or a computational protein structure or homology model can be performed using the predicted pose as input.

Accordingly, embodiments of the present invention provide an accurate prediction of binding modes between a target molecule (e.g., a biopolymer such as a protein or nucleic acid) and its ligand(s) (any molecule that potentially binds to the target), which is an important step of computational and structure-based drug design. The one or more embodiments of the present invention accordingly provide a practical application and an improvement to at least the field of computational and structure-based drug design, and to computational chemistry in general. As described herein, one or more embodiments of the present invention improve the prediction of target-ligand binding modes by combining predictions from small molecule docking tools and target-ligand cheminformatics within a unified deep learning framework.

Comparisons of existing techniques with one or more embodiments of the present invention indicate that one or more embodiments of the present invention provide an improved accuracy in determining the binding mode prediction by using deep-learning with the combined data being used as input features as described herein. Accuracy of the techniques can be gauged, for example, by computing the ligand RMSD of high ranked poses from one or more embodiments of the present invention with experimental structures. A ligand RMSD less than 2.0 Angstroms is considered accurate in the field.

One or more embodiments of the present invention facilitate combining informatic, deep learning-generated features of a ligand and corresponding molecular target-ligand complex with features output by docking programs into a deep learning framework for binding mode prediction, constituting a novel aspect of our invention.

The binding mode prediction provided by one or more embodiments of the present invention can guide virtual screening and later stages of the drug discovery workflow(s).

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instruction by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments described. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein.

What is claimed is:

1. A computer-implemented method comprising:
generating, by a ligand bond graph generator, a first graph based on bond connectivity within a ligand molecule that is specified as input;
generating, by a ligand-protein graph generator, a second graph based on a contact map of the ligand molecule and a target molecule that is specified as another input;
receiving docking prediction metrics for the ligand molecule and the target molecule;
inputting, to a deep neural network, as input features, the first graph, the second graph, and the docking prediction metrics; and
determining, using the deep neural network, a binding mode prediction that characterizes a set of potential interactions between the ligand molecule and the target molecule.

2. The computer-implemented method of claim 1, wherein the docking prediction metrics are computed by a docking program.

3. The computer-implemented method of claim 1, wherein the binding mode prediction is extracted from an output layer of the deep neural network.

4. The computer-implemented method of claim 1 further comprising generating the contact map that comprises a graph of ligand sites and target molecule sites that are in contact based on a 3-dimensional representation of the ligand molecule and the target molecule, a graph edge from the contact map connects a ligand site and a target molecule site.

5. The computer-implemented method of claim 4, wherein the ligand site and the target molecule site are in contact based on a distance between the ligand site and the target molecule site being below a predetermined threshold, wherein the graph edge is weighted according to the distance between the ligand site and the target molecule site connected by the graph edge.

6. The computer-implemented method of claim 1 further comprising generating an internal representation of the first graph prior to inputting the first graph into the deep neural network.

7. The computer-implemented method of claim 6 further comprising generating an internal representation of the second graph prior to inputting the second graph into the deep neural network.

8. The computer-implemented method of claim 1 further comprising using the binding mode prediction for lead optimization and/or lead finding by predicting binding modes of one or more ligand molecules with the target molecule.

9. A system comprising:
a memory;
a processor coupled with the memory, the processor configured to perform a method for providing binding mode predictions for one or more ligand molecules and a target molecule, the method comprising:
generating a first graph based on bond connectivity within a ligand molecule that is specified as input;
generating a second graph based on a contact map of the ligand molecule and a target molecule that is specified as another input;
receiving docking prediction metrics for the ligand molecule and the target molecule;
inputting, to a deep neural network, as input features, the first graph, the second graph, and the docking prediction metrics; and
determining, using the deep neural network, a binding mode prediction that indicates a set of potential interactions between the ligand molecule and the target molecule.

10. The system of claim 9, wherein the docking prediction metrics are computed by a docking program.

11. The system of claim 9, wherein the binding mode prediction is extracted from an output layer of the deep neural network.

12. The system of claim 9 further comprising generating the contact map that comprises a graph of ligand sites and target molecule sites that are in contact based on a 3-dimensional representation of the ligand molecule and the target molecule.

13. The system of claim 12, wherein the ligand site and the target molecule site are in contact based on a distance between the ligand site and the target molecule site being below a predetermined threshold, wherein the graph edge is weighted according to the distance between the ligand site and the target molecule site connected by the graph edge.

14. The system of claim 9 further comprising:
generating an internal representation of the first graph prior to inputting the first graph into the deep neural network; and
generating an internal representation of the second graph prior to inputting the second graph into the deep neural network.

15. The system of claim 9 further comprising using the binding mode prediction for lead optimization and/or lead finding by predicting binding modes of one or more ligand molecules with the target molecule.

16. A computer program product comprising a memory storage device having computer executable instructions stored therein, the computer executable instructions when executed by a processor cause the processor to perform a method for providing binding mode predictions for one or more ligand molecules and a target molecule, the method comprising:
generating a first graph based on bond connectivity within a ligand molecule that is specified as input;
generating a second graph based on a contact map of the ligand molecule and a target molecule that is specified as another input;
receiving docking prediction metrics for the ligand molecule and the target molecule;
inputting, to a deep neural network, as input features, the first graph, the second graph, and the docking prediction metrics; and
determining, using the deep neural network, a binding mode prediction that characterizes a set of potential interactions between the ligand molecule and the target molecule.

17. The computer program product of claim 16, wherein the docking prediction metrics are computed by a docking program.

18. The computer program product of claim 16, wherein the binding mode prediction is extracted from an output layer of the deep neural network.

19. The computer program product of claim 16, wherein the method performed by the processor further comprises generating the contact map that comprises a graph of ligand sites and target molecule sites that are in contact based on a 3-dimensional representation of the ligand molecule and the target molecule, wherein a ligand site and a target molecule site are determined to be in contact based on a distance between the ligand site and the target molecule site being below a predetermined threshold.

20. The computer program product of claim 16, wherein the method performed by the processor further comprises:
generating an internal representation of the first graph prior to inputting the first graph into the deep neural network; and
generating an internal representation of the second graph prior to inputting the second graph into the deep neural network.
